(19) United States
(12) Patent Application Publication
(10) Pub. No.: US 2004/0137583 A1
(43) Pub. Date: Jul. 15, 2004

(54) Method for producing high OD phycoerythrin

(76) Inventors: Young-Meng Chiang, Taipei City (TW); Hong-Nong Chou, Taipei City (TW); Jiunn-Ming Jeng, Sinjhuang City (TW); Chih-Yi Lu, Jhongpu Township (TW)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/713,029

(22) Filed: Nov. 17, 2003

(30) Foreign Application Priority Data
Nov. 21, 2002 (TW) 91133928
Nov. 21, 2002 (TW) 91133929
Nov. 21, 2002 (TW) 91133930
Nov. 21, 2002 (TW) 91133931

(51) Int. Cl. C12P 17/18

(52) U.S. Cl. 435/119

(57) ABSTRACT

A new method for producing phycoerythrin with high optical density [OD] from the group selected from an algae whose life cycle has sexual reproduction, asexual reproduction, and vegetative propagation, such as Galaxaura oblongata, Halymenia ceylanica, Helminthocladia australis, and Porphyra dentata.

### BACKGROUND OF THE INVENTION

1. Field of the Invention

The present invention relates to a new method for producing phycoerythrin with high optical density [OD], and more particularly to a new method for producing phycoerythrin with high optical density [OD] from the group consisting of Galaxaura oblongata, Halymenia ceylanica, Helminthocladia australis, and Porphyra dentata.

2. Description of the Prior Art

Natural pigment proteins from plants are safe when used in food and drink. The pigments are stable in mild heat, and acidic or basic solutions. Therefore they can be utilized in food and cosmetics as coloring agent. In additional, the pure form of the pigment can be used in fluorescent labeling of antibodies that were applied as diagnostic agent in immunological, clinical, cell biological and biochemical research.

Phycocyanin and phycoerythrin are two currently used natural pigment proteins, and have been applied in many fields. As the major raw material for phycocyanin is easy-growing blue-green algae such as Spirulina and Microcystis and a large number of methods of algae cultivation and phycocyanin preparation therefrom have been developed, the supply of phycocyanin does not cause a problem. However, the quantity of phycoerythrin is still few and the price is high due to the shortage of raw material available and the difficulty in processing for the commercial production of phycoerythrin.

Most of the phycoerythrin is extracted from red algae thalli such as Porphyra and Ceramium, and only a little amount of phycoerythrin are extracted from Porphyridium, which is now available from tank cultivation.

Although increasing amount of wild red algae and cultivated Porphyra are utilized as raw material for phycoerythrin, most of them contain a high gel content, making the extraction of phycoerythrin from them be very difficult, especially for dried algae. Furthermore, the quantity and quality of wild algae are apt to be influenced by the seasons and the ambient temperature. These elements make the production of phycoerythrin from wild and cultivated Porphyra even more difficult.

Extracting phycoerythrin from Porphyridium also has its difficulties, because the collection of single-cell is usually labor-intensive as well as time-consuming and the soluble polysaccharide secreted during the cultivation of algae will deter the cell collection and influence the extraction of phycoerythrin.

To solve the above problems, the process for preparing phycoerythrin from Bangia atropurpurea and Porphyra angusta is disclosed in the U.S. Pat. No. 5,358,858. Because the filamentous plants thereof do not contain gel and can thus be maintained under some controlled conditions such as culture medium, temperature, illuminance, and daily illuminating period. Phycoerythrin can be extracted from the filamentous plants easily. The process of the above invention includes the following steps:

1. Mature Bangia atropurpurea or Porphyra angusta thalli are collected from sea and washed with sterilized seawater. After a short time of air-drying, they are placed into culture medium (SWM-III medium). After a few hours, spores will be released from the Bangia atropurpurea or Porphyra angusta thalli. The released spores are then removed from original medium and placed in a growth chamber wherein the temperature, illuminance, and daily illuminating period are ambient temperature, 1000 lux-4000 lux and 10-16 hours.

2. After the spores germinate to branched filaments, the filaments are transferred to SWM-III medium-containing flasks, and cultivated in the above condition until they form colonies. The filamentous colonies are then cut into small segments using sterilized grinder and moved to a larger space, such as a tank, in order to facilitate the further growth. After they are transferred into a larger space, more filaments are generated. The filamentous colonies are cut again for further growth until the required amount is acquired. Note that when the filamentous colonies are cultivated in a large tank, fresh air (300 ml air/min) must be supplied to the tank. The filaments are then collected and filtered by a net of 100-400 mesh. The culture medium can be recovered and reused.

3. The collected and filtered Bangia atropurpurea or Porphyra angusta filaments are then fast dried in vacuum or by warm air and ground into powder. The powder is added to a solution of phosphate or water and mixed completely. Debris are removed by centrifugation to obtain a clear-red pigment solution. Crude phycoerythrin can then be obtained by adding (NH.sub.4).sub.2 SO.sub.4 to make it as 20%-30% saturated solution to remove unnecessary proteins, followed by sedimentation with 60%-65% (NH.sub.4).sub.2 SO.sub.4 saturated solution. The phycoerythrin obtained has an OD.sub.565/OD.sub.280 of 1.4-1.6 and become food-grade and cosmetics-usable pigments.

4. The crude precipitated phycoerythrin can be further purified by gel filtration chromatography. For example, after purifying with Sephadex G200 chromatography once, the OD.sub.565/OD.sub.280 ratio of the produced phycoerythrin can reach to 3.3-3.7. After repeated purification process, the OD.sub.565/OD.sub.280 ratio can reach to 5.1-5.2. The purity of the phycoerythrin is about 99% when tested with SDS electrophoresis. This indicates that the phycoerythrin produced by the process of the invention can be used as reagents for immunoassay.

Owing to phycoerythrin with the 5.1-5.2 value of OD.sub.565/OD.sub.280 ratio is obtained through the complex purification processes twice at step 4. It increases the manufacturing cost and time. Therefore, we need to find a new method that uses the filaments of other plants to produce directly phycoerythrin with high OD from the first clear-red pigment solution at step 3.

### SUMMARY OF THE INVENTION

In the light of the state of the art described above, it is an object of the present invention to provide a new method for producing phycoerythrin with high optical density which is immune to the problems of the conventional process for preparing phycoerythrin from Bangia atropurpurea and Porphyra angusta described above.

It is another object of this invention to provide a new method for producing phycoerythrin with high optical density that the unit weight of the algae selected from the group consisting of Galaxaura oblongata, Halymenia ceylanica, Helminthocladia australis, and Porphyra dentata has plenty of weight of phycoerythrin to reduce the manufacturing cost.

It is a further object of this invention to provide a new method for producing phycoerythrin with high optical density that the first clear-red pigment solution of the algae selected from the group consisting of Galaxaura oblongata, Halymenia ceylanica, Porphyra dentata, and Helminthocladia australis has high OD phycoerythrin to reduce the manufacturing step.

In view of the above and other objects which will become apparent as the description proceeds, there is provided according to a general aspect of the present invention a new method for producing phycoerythrin with high optical density (OD), which comprises the following steps: cultivating a gametophyte with mature tetrasporangia in a medium to obtain tetraspores therefrom; cultivating said tetraspores in a condition that the temperature, light intensity and light/dark ratio are respectively 15-30° C., 500 lux-6000 lux and above 10:14 to germinate filaments; collecting said cultivated filaments; adding said cultivated filaments to a liquid solution with the pH value of 5-10; obtaining a clear-red pigment protein solution containing phycoerythrin by centrifuging said liquid solution at 6000 rpm for 10 minutes at 4° C.; and salting out the gel-form phycoerythrin concentrate from said clear-red pigment protein solution, wherein said gametophyte selected from an algae whose life cycle has sexual reproduction, asexual reproduction, and vegetative propagation.

Base on the idea described above, wherein said algae is selected from the group consisting of Galaxaura oblongata, Halymenia ceylanica, Helminthocladia australis, and Porphyra dentata.

Base on the aforementioned idea, wherein chromatography spectrogram at 565 nm of phycoerythrin extracted from said cultivated filaments of Galaxaura oblongata carpospores measured by High Performance Liquid Chromatography (HPLC) is shown as the FIG. 7B.

Base on the idea described above, wherein chromatography spectrogram at 565 nm of phycoerythrin extracted from said cultivated filaments of Halymenia ceylanica carpospores measured by High Performance Liquid Chromatography (HPLC) is shown as the FIG. 8B.

Base on the aforementioned idea, wherein chromatography spectrogram at 565 nm of phycoerythrin extracted from said cultivated filaments of Helminthocladia australis carpospores measured by High Performance Liquid Chromatography (HPLC) is shown as the FIG. 9B.

Base on the idea described above, wherein chromatography spectrogram at 565 nm of phycoerythrin extracted from said cultivated filaments of Porphyra dentata carpospores measured by High Performance Liquid Chromatography (HPLC) is shown as the FIG. 10B.

Base on the aforementioned idea, wherein said medium is a SWM-III medium.

Base on the idea described above, wherein said SWM-III medium is an inorganic SWM-III medium.

Base on the aforementioned idea, wherein the step of cultivating said tetraspores to germinate filaments further comprises that breaking up said filaments into minute segments and cultivating them in a larger tank in the same condition until the cultivated filaments grow to the required amounts, wherein said tank is supplied with the fresh air for keeping said minute segments to be suspended in the medium.

Base on the idea described above, wherein the better temperature, light intensity and light/dark ratio of said condition are respectively 20° C., 2000 lux, and 12:12.

Base on the aforementioned idea, wherein the step of collecting said cultivated filaments further comprises that collecting said cultivated filaments by a net of 20-400 mesh, drying said cultivated filaments, and grinding said cultivated filaments into powder.

Base on the idea described above, wherein the method of drying said cultivated filaments is selected from the group consisting of the vacuum method or the warm air method.

Base on the aforementioned idea, wherein said liquid solution consists of water and potassium phosphate.

Base on the idea described above, wherein the step of salting out the gel-form phycoerythrin further comprises that adding the 20% solution of (NH.sub.4).sub.2 SO.sub.4 to said clear-red pigment protein solution, and centrifuging said clear-red pigment protein solution at 6000 rpm for 10 minutes at 4° C. for separating the unwanted proteins to obtain a purer pigment protein solution.

Base on the idea described above, wherein the step of salting out the gel-form phycoerythrin further comprises that adding the 60-65% solution of (NH.sub.4).sub.2 SO.sub.4 to said purer pigment protein solution, and centrifuging said purer pigment protein solution at 6000 rpm for 10 minutes at 4° C. to obtain the gel-form phycoerythrin concentrate.

Base on the aforementioned idea, wherein the step of salting out the gel-form phycoerythrin further comprises that dialyzing said gel-form phycoerythrin concentrate and purifying the phycoerythrin by gel filtration therefrom.

Base on the idea described above, wherein the gel filtration is a Sephadex G200 gel filtration.

Base on the aforementioned idea, wherein the step of salting out the gel-form phycoerythrin further comprises that purifying said gel-form phycoerythrin concentrate by ultrafiltration therefrom.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 shows chromatography spectrogram of absorption and emission of phycoerythrin measured by High Performance Liquid Chromatography (HPLC);

FIG. 2 shows the life cycle of Bangia atropurpurea;

FIG. 3 shows the life cycle of Porphyra angusta;

FIG. 4 shows the life cycle of Nemalion;

FIGS. 5A˜5C show the chromatography spectrogram of phycoerythrin extracted from Bangia atropurpurea measured by HPLC at 280 nm, 565 nm, and 615 nm;

FIGS. 6A˜6C show the chromatography spectrogram of phycoerythrin extracted from Porphyra angusta measured by HPLC at 280 nm, 565 nm, and 615 nm;

FIGS. 7A˜7C show the chromatography spectrogram of phycoerythrin extracted from Galaxaura oblongata measured by HPLC at 280 nm, 565 nm, and 615 nm;

FIGS. 8A˜8C show the chromatography spectrogram of phycoerythrin extracted from Halymenia ceylanica measured by HPLC at 280 nm, 565 nm, and 615 nm;

FIGS. 9A˜9C show the chromatography spectrogram of phycoerythrin extracted from Helminthocladia australis measured by HPLC at 280 nm, 565 nm, and 615 nm; and

FIGS. 10A˜-10C show the chromatography spectrogram of phycoerythrin extracted from Porphyra dentata measured by HPLC at 280 nm, 565 nm, and 615 nm.

### DESCRIPTION OF THE PREFERRED EMBODIMENT

Some sample embodiments of the present invention will now be described in greater detail. Nevertheless, it should be recognized that the present invention can be practiced in a wide range of other embodiments besides those explicitly described, and the scope of the present invention is expressly not limited except as specified in the accompanying claims.

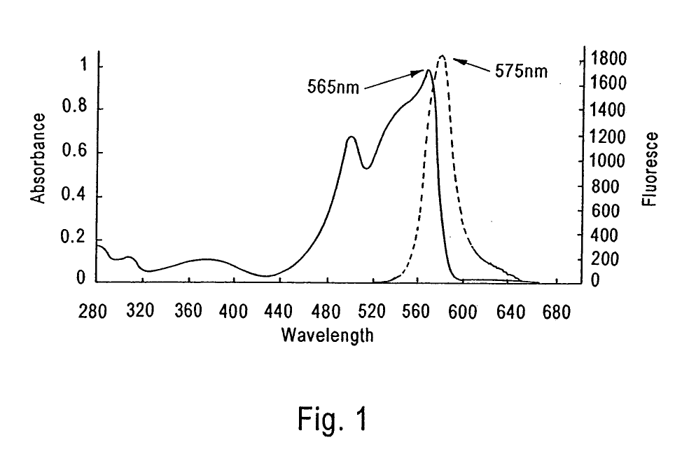

Phycobiliproteins are the water-soluble fluorescent pigment proteins from algae. They can be widely used in fluorescent labeling of antibodies that were applied as diagnostic agents owing to their special fluorescent properties. Phycoerythrin has the highest fluorescent intensity among Phycobiliproteins, so it is adopted in many fluorescent tests. Chromatography spectrogram of absorption and emission of phycoerythrin measured by High Performance Liquid Chromatography (HPLC) is shown as FIG. 1. The chromatography conditions are as below:

HPLC column: HYDROCELL DEAE NP10

Column size: 50*4.6 mm

Buffer A: 10 mMK-PBS pH6.0

Buffer B:10 mMk-PBS, 0.5M NaCl pH6.0

Gradient:0% Buffer B→12 min→50% Buffer B

Detection:565 nm

Flow rate: 1 ml/min

HPLC is mainly constituted by pump accessories, filters, detectors, and recorder.

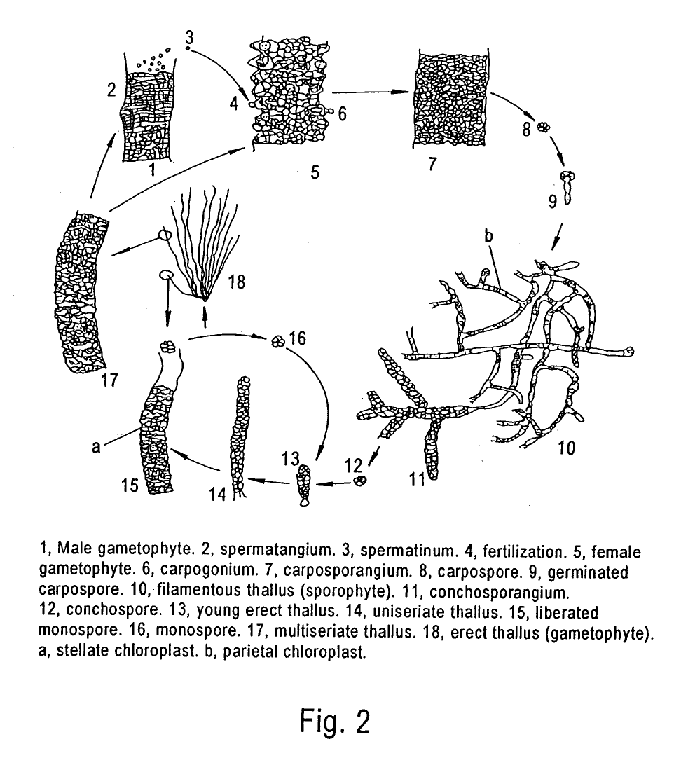

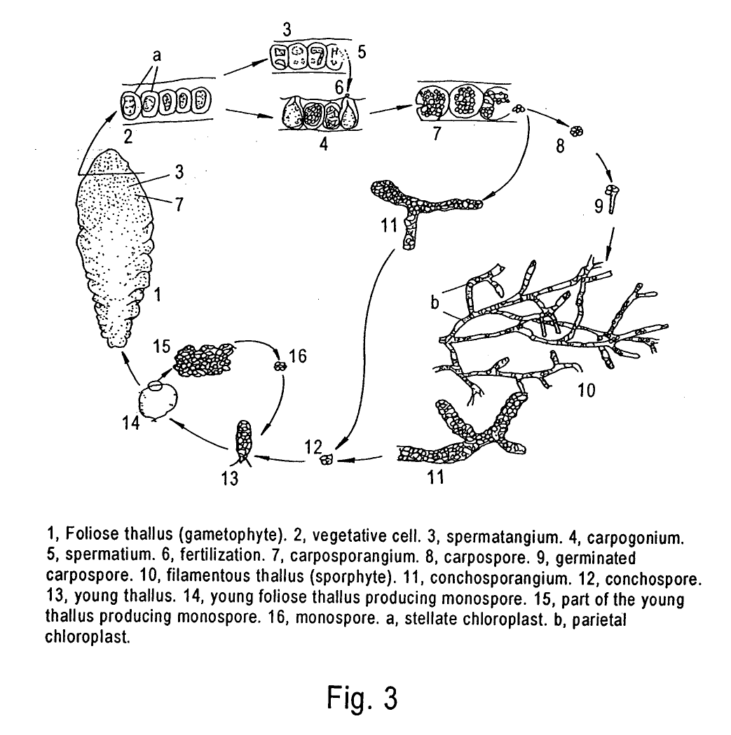

There are alternation of generations of sexual reproduction and asexual reproduction in the life cycles of Bangia atropurpurea and Porphyra angusta, as shown in FIGS. 2 and 3. A mature male gametophyte produces spermatiums in the spermatangiums. These spermatiums are released into the water and are carried by currents to the carpogoniums of female gametophyte. A mature carpogonium produces carpospore to form filamentous thallus (sporophyte) with conchosporangia, and the conchospores are released from the mature conchosporangium to form young erect thalli. After a time, the young erect thalli will release the monospores to form the new young erect thalli repeatedly or the erect thalli (gametophytes) in the proper conditions. So the alternation of generations with sexual reproduction and asexual reproduction are continue.

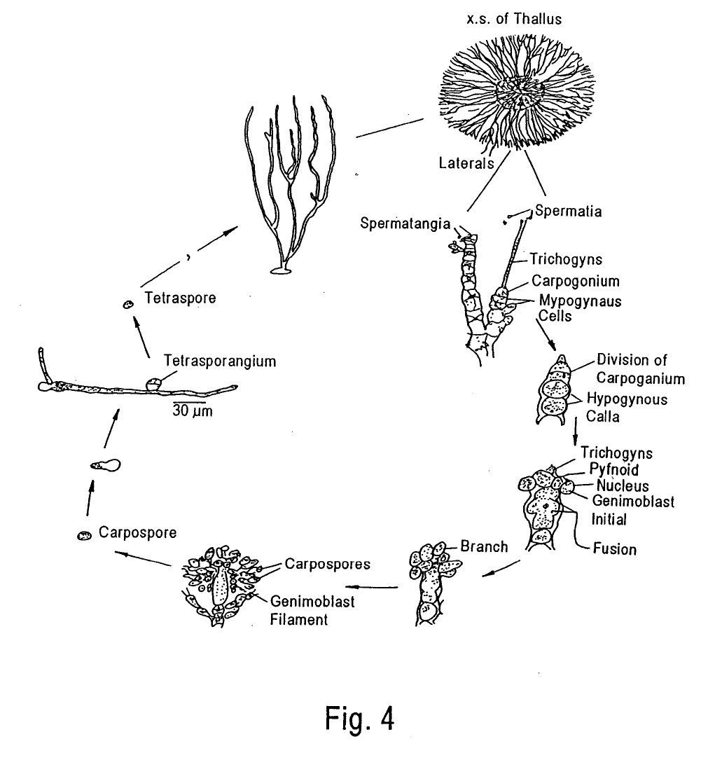

There are alternation of generations of sexual reproduction, asexual reproduction and vegetative propagation in the life cycles of other algae, such as Nemalion shown in FIG. 4. A mature carpogonium produces carpospore to form tetrasporophyte with tetrasporangia, and the tetraspores are released from the mature tetrasporangium to form filamentous thallus. There are different properties between the filamentous thallus of tetraspore and the filamentous thallus of carpospore. Similarly, the filamentous thallus of tetraspore do not contain gel and can thus be maintained under some controlled conditions such as culture medium, temperature, illuminance, and daily illuminating period. Phycoerythrin with high OD extracted from the filamentous thallus of tetraspore easily is disclosed in this invention. The process of the prevent invention includes the following steps:

1. The gametophytes with mature tetrasporangia selected from the algae whose life cycle has sexual reproduction, asexual reproduction, and vegetative propagation, such as Galaxaura oblongata, Halymenia ceylanica, Helminthocladia australis, and Porphyra dentata, are collected and washed with sterilized water. After a short time of air-drying, they are placed into a culture medium (inorganic SWM-III medium). After a few hours, tetraspores will be released from the algae. The released tetraspores are then removed from original medium and placed in a growth chamber wherein the temperature, illuminance, light/dark ratio and daily illuminating period are respectively 15-30° C., 500 lux-6000 lux, above 10:14, and 10-16 hours every day. But the better temperature, light intensity and light/dark ratio are respectively 20° C., 2000 lux, and 12:12.

2. After the tetraspores germinate to branched filaments, the filaments are transferred to inorganic SWM-III medium-containing flasks, and cultivated in the above condition until they form colonies. The filamentous colonies are then cut into small segments using sterilized grinder and moved to a larger space, such as a tank, in order to facilitate the further growth. After they are transferred into a larger space, more filaments are generated. The filamentous colonies are cut again for further growth until the required amount is acquired. Note that when the filamentous colonies are cultivated in a large tank, fresh air (300 ml air/min) must be supplied to the tank to keep the colonies to be suspended in the medium. The filaments are then collected and filtered by a net of 20-400 mesh. The culture medium can be recovered and reused.

3. The collected and filtered the filaments are then fast dried in vacuum or by warm air and ground into powder. The powder is added to a solution with the pH value of 5-10 of potassium phosphate or water and mixed completely. Debris are removed by centrifugation at 6000 rpm for 10 minutes at 4° C. to obtain a clear-red pigment solution. Crude phycoerythrin can then be obtained by adding (NH.sub.4).sub.2 SO.sub.4 to make it as 20%-30% saturated solution, centrifuging the saturated solution at 6000 rpm for 10 minutes at 4° C. to remove unnecessary proteins, adding 60%-65% (NH.sub.4).sub.2 SO.sub.4 saturated solution by sedimentation, and centrifuging the solution at 6000 rpm for 10 minutes at 4° C. to obtain the crude phycoerythrin with high OD to become food-grade and cosmetics-usable pigments. There are the OD values in Table 1 for the six algae that comprise Bangia atropurpurea, Porphyra angusta, Galaxaura oblongata, Halymenia ceylanica, Helminthocladia australis, and Porphyra dentata. It should be better for a phycoerythrin with higher OD A₅₆₅/A₂₈₀ and lower OD A₆₁₅/A₅₆₅.

TABLE 1

| | algae | | | | | |
|---|---|---|---|---|---|---|
| | Ba | Pa | Go | Hc | Ha | Pd |
| RPE (mg)/algae (g) | 53.5 | 38.89 | 57.74 | 46.59 | 48.1 | 44.98 |
| OD A₅₆₅/A₂₈₀ | 1.40 | 1.54 | 2.66 | 1.44 | 2.34 | 1.96 |
| OD A₆₁₅/A₅₆₅ | 0.19 | 0.53 | 0.14 | 0.10 | 0.15 | 0.21 |

RPE: a phycoerythrin protein with maximum UV absorbing wavelength 566 nm and fluorescence emission wavelength 575 nm.
Ba: Bangia atropurpurea
Pa: Porphyra angusta
Go: Galaxaura oblongata
Hc: Halymenia ceylanica
Ha: Helminthocladia australis
Pd: Porphyra dentata

4. The crude precipitated phycoerythrin can be further purified by gel filtration chromatography or ultrafiltration. For example, after purifying with Sephadex G200 chromatography once, the OD.sub.565/OD.sub.280 ratio of the produced phycoerythrin can reach to 4.5. After repeated purification process, the OD.sub.565/OD.sub.280 ratio of phycoerythrin can reach to 5.3. The purity of the phycoerythrin is about 99% when tested with SDS electrophoresis. This indicates that the phycoerythrin produced by the process of the invention can be used as reagents for immunoassay.

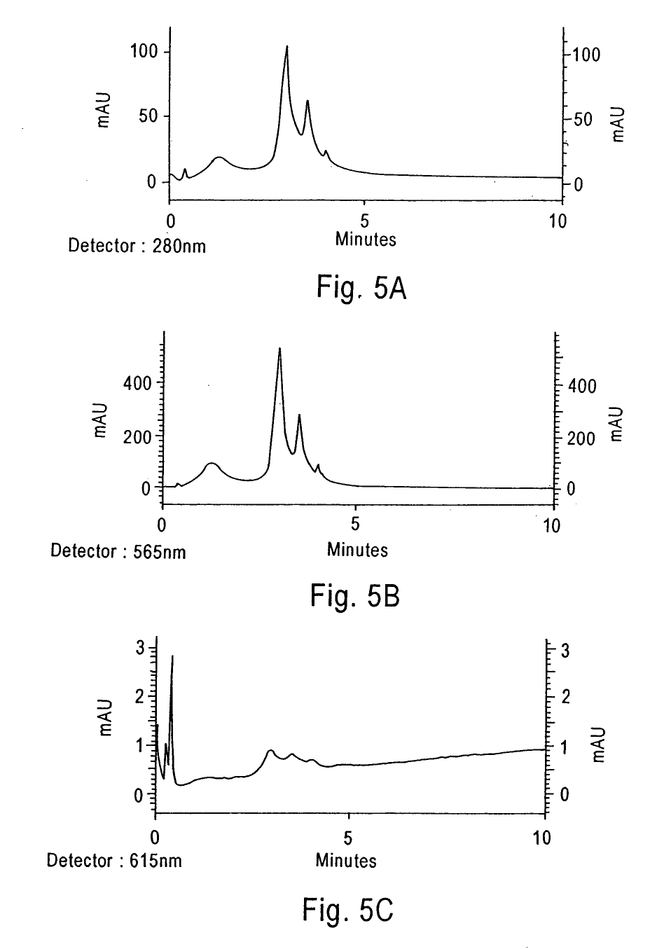

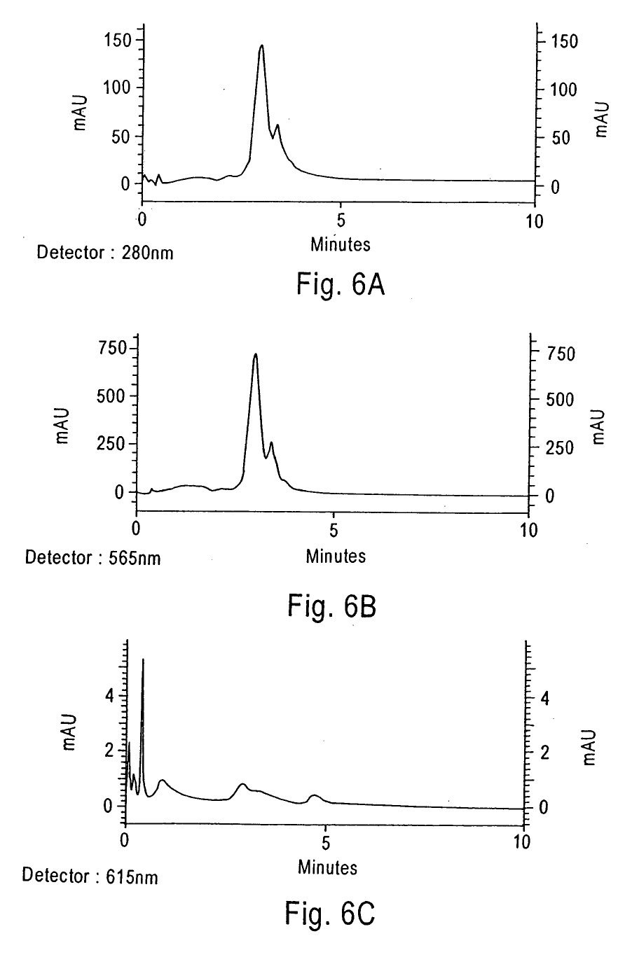

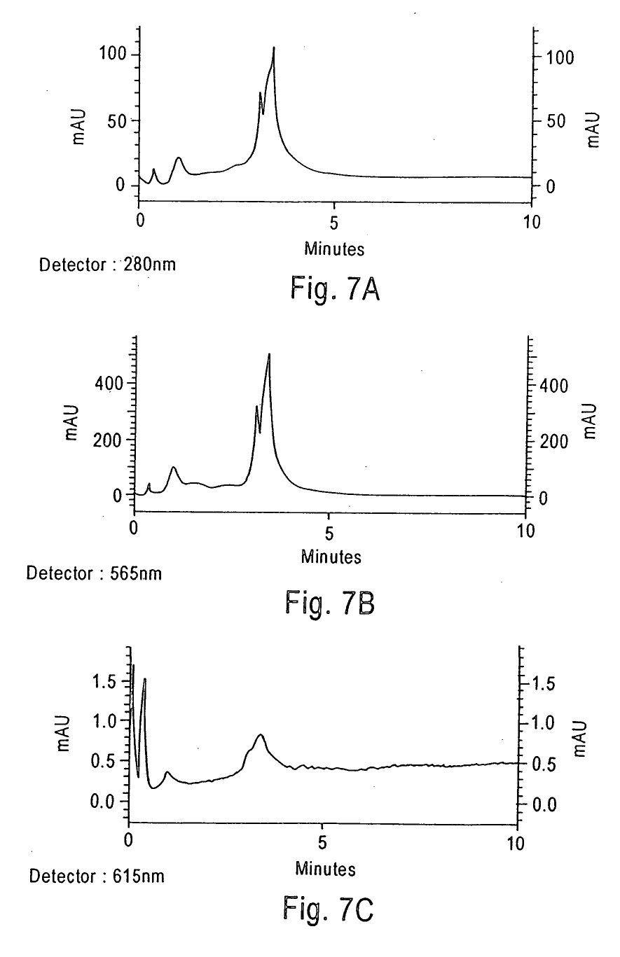

The chromatography spectrogram of phycoerythrin extracted from Bangia atropurpurea and Porphyra angusta measured by HPLC at 280 nm, 565 nm, and 615 nm are shown in FIGS. 5A-6C. FIGS. 7A-10C show the chromatography spectrogram of phycoerythrin extracted from Galaxaura oblongata, Halymenia ceylanica, Helminthocladia australis, and Porphyra dentata measured by HPLC at 280 nm, 565 nm, and 615 nm respectively. The phycoerythrin extracted from each alga has the special chromatography spectrogram, so we can know easily the source of phycoerythrin by HPLC.

Although the specific embodiment has been illustrated and described, it will be obvious to those skilled in the art that various modifications may be made without departing from what is intended to be limited solely by the appended claims.

What is claimed is:

1. A new method for producing phycoerythrin with high optical density (OD), comprising the following steps:
cultivating a gametophyte with mature tetrasporangia in a medium to obtain tetraspores therefrom;
cultivating said tetraspores in a condition that the temperature, light intensity and light/dark ratio are respectively 15-30′, 500 lux-6000 lux and above 10:14 to germinate filaments;
collecting said cultivated filaments;
adding said cultivated filaments to a liquid solution with the pH value of 5-10;
obtaining a clear-red pigment protein solution containing phycoerythrin by centrifuging said liquid solution at 6000 rpm for 10 minutes at 4° C.; and
salting out the gel-form phycoerythrin concentrate from said clear-red pigment protein solution, wherein said gametophyte selected from an algae whose life cycle has sexual reproduction, asexual reproduction, and vegetative propagation.

2. The method according to claim 1, wherein said algae is selected from the group consisting of Galaxaura oblongata, Halymenia ceylanica, Helminthocladia australis, and Porphyra dentata.

3. The method according to claim 2, wherein chromatography spectrogram at 565 nm of phycoerythrin extracted from said cultivated filaments of Galaxaura oblongata carpospores measured by High Performance Liquid Chromatography (HPLC) is shown as the FIG. 7B.

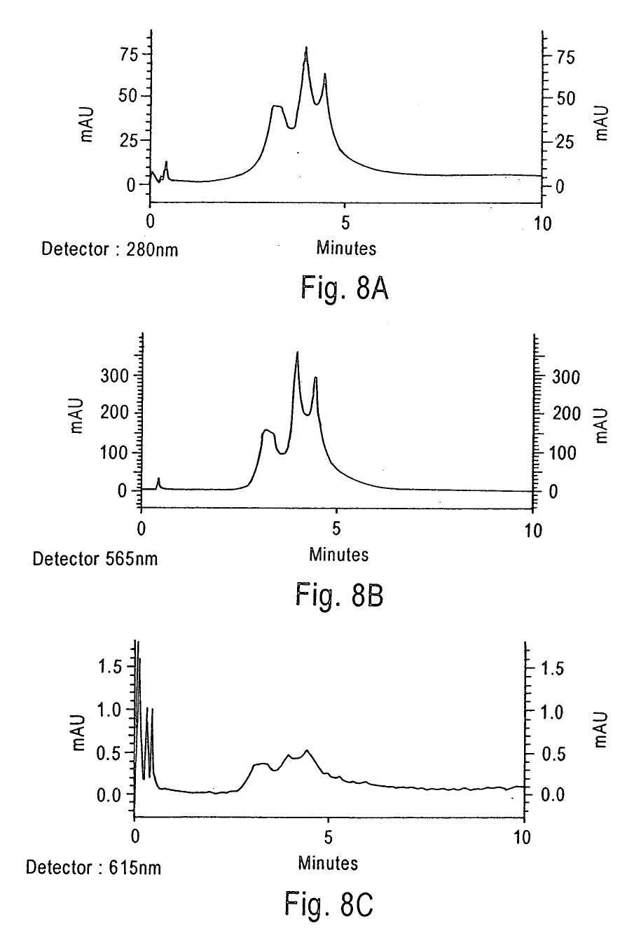

4. The method according to claim 2, wherein chromatography spectrogram at 565 nm of phycoerythrin extracted from said cultivated filaments of Halymenia ceylanica carpospores measured by High Performance Liquid Chromatography (HPLC) is shown as the FIG. 8B.

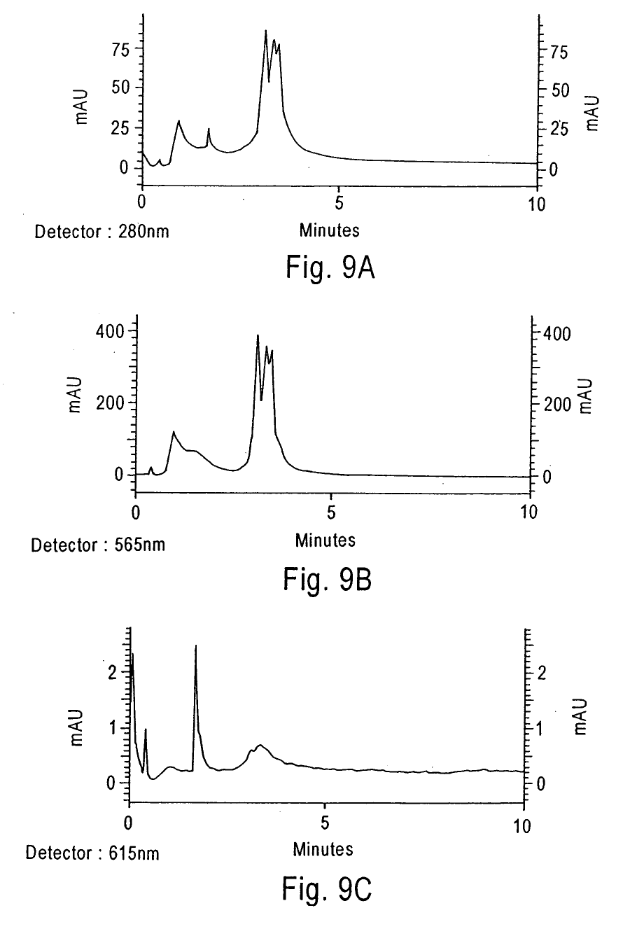

5. The method according to claim 2, wherein chromatography spectrogram at 565 nm of phycoerythrin extracted from said cultivated filaments of Helminthocladia australis carpospores measured by High Performance Liquid Chromatography (HPLC) is shown as the FIG. 9B.

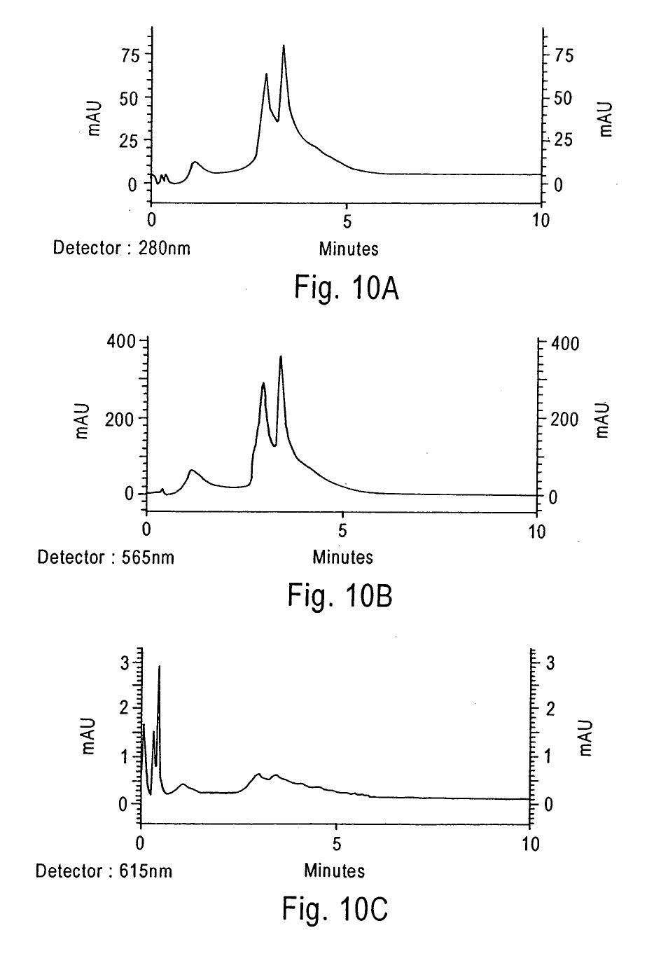

6. The method according to claim 2, wherein chromatography spectrogram at 565 nm of phycoerythrin extracted from said cultivated filaments of Porphyra dentata carpospores measured by High Performance Liquid Chromatography (HPLC) is shown as the FIG. 10B.

7. The method according to claim 1, wherein said medium is a SWM-III medium.

8. The method according to claim 7, wherein said SWM-III medium is an inorganic SWM-III medium.

9. The method according to claim 1, wherein the step of cultivating said tetraspores to germinate filaments further comprises:
breaking up said filaments into minute segments and cultivating them in a larger tank in the same condition until the cultivated filaments grow to the required amounts, wherein said tank is supplied with the fresh air for keeping said minute segments to be suspended in the medium.

10. The method according to claim 1, wherein the better temperature, light intensity and light/dark ratio of said condition are respectively 20° C., 2000 lux, and 12:12.

11. The method according to claim 1, wherein the step of collecting said cultivated filaments further comprises:
collecting said cultivated filaments by a net of 20-400 mesh;
drying said cultivated filaments; and
grinding said cultivated filaments into powder.

12. The method according to claim 1, wherein the method of drying said cultivated filaments is selected from the group consisting of the vacuum method or the warm air method.

13. The method according to claim 1, wherein said liquid solution consists of water and potassium phosphate.

14. The method according to claim 1, wherein the step of salting out the gel-form phycoerythrin further comprises:
adding the 20% solution of (NH.sub.4).sub.2 SO.sub.4 to said clear-red pigment protein solution; and
centrifuging said clear-red pigment protein solution at 6000 rpm for 10 minutes at 4° C. for separating the unwanted proteins to obtain a purer pigment protein solution.

15. The method according to claim 14, wherein the step of salting out the gel-form phycoerythrin further comprises:
adding the 60-65% solution of (NH.sub.4).sub.2 SO.sub.4 to said purer pigment protein solution; and
centrifuging said purer pigment protein solution at 6000 rpm for 10 minutes at 4° C. to obtain the gel-form phycoerythrin concentrate.

16. The method according to claim 15, wherein the step of salting out the gel-form phycoerythrin further comprises:
dialyzing said gel-form phycoerythrin concentrate and purifying the phycoerythrin by gel filtration therefrom.

17. The method according to claim 16, wherein the gel filtration is a Sephadex G200 gel filtration.

18. The method according to claim 15, wherein the step of salting out the gel-form phycoerythrin further comprises:
purifying said gel-form phycoerythrin concentrate by ultrafiltration therefrom.
